(19) United States
(12) Patent Application Publication
(10) Pub. No.: US 2005/0283081 A1
(43) Pub. Date: Dec. 22, 2005

(54) Non-invasive apparatus for measuring physiological variables

(75) Inventors: Jia-Lung Lin, Ji-An Township (TW); Bih-Chern Lin, Taichung City (TW); Yang-Hwei Tsuang, Taipei City (TW); Fon-Yih Tsuang, Taipei City (TW)

Correspondence Address: John S. Egbert;Harrison & Egbert, 7th Floor, 412 Main Street, Houston, TX, 77002, US

(73) Assignees: Jia-Lung Lin, Ji-An Township (TW); Bih-Chern Lin, Taichung City (TW)

(21) Appl. No.: 11/128,476

(22) Filed: May 13, 2005

(30) Foreign Application Priority Data
Jun. 16, 2004 (TW) 093117307

(51) Int. Cl. A61B 6/00; A61B 5/00

(52) U.S. Cl. 600/474; 600/549

(57) ABSTRACT

The present invention provides a non-invasive apparatus for continuously measuring a plurality of physiological variables from a user's ear. The non-invasive apparatus includes a body, a temperature detector positioned on the body, a plastic housing encapsulating the body, a light emitting device for emitting a light beam to a tragus of the user's ear, and a light receiver for receiving the light beam penetrating through the tragus. The temperature detector can measure the user's body temperature from a tympanic membrane. The light emitting device incorporating the light receiver can measure the blood oxygen saturation of the user by detecting the energy loss of the light beam due to the penetration through the tragus. The plastic housing includes an awl-shaped portion capable of inserting into an auditory meatus of the user's ear and a protrusion capable of engaging with a triangular fossa of the user's ear.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates to a non-invasive apparatus for measuring physiological variables, and more particularly, to a non-invasive apparatus for continuous measuring a plurality of physiological variables from a user's ear.

### BACKGROUND OF THE INVENTION

In the diagnosis process, the clinical thermometer does great favor to physicians. Among all positions to be measured, the temperature of tympanic membrane is the best indication of the body temperature rather than those of oral cavity, rectum or armpit. The temperature of the tympanic membrane is measured by detecting the infrared radiation emitted from tympanic membrane. Consequently, the infrared ear thermometer is able to measure and display the ear temperature efficiently within one or two seconds, and is widely used by hospitals, clinics or family, gradually replacing the traditional mercury thermometer.

While measuring the infrared radiation in an auditory meatus, the detector of the ear thermometer has to be inserted into the external auditory meatus of a patient before the infrared radiation can be measured accurately, and the body temperature can then be derived based on the infrared radiation. However, the insertion of the detector into the external auditory meatus may cause the patient's discomforts such as the sense of a foreign matter, so the traditional detector is only allowed to stay in the external auditory meatus of the patient for a very short while to mitigate his discomforts. In other words, the traditional ear thermometer is not suitable to be fixed on the patient's ear for gathering consecutive body temperature data of the patient.

A traditional pulse oxymeter uses a non-invasive optical detector to consecutively measure the blood oxygen saturation of a subject such as a patient's body. Particularly, the pulse oxymeter uses the diverse characteristics of the optical absorbance between the hemoglobin without oxygen (Hb) and the hemoglobin with oxygen (HbO₂), and derives the blood oxygen saturation in a human body from the absorbency of the Hb and HbO₂. However, one disadvantage of the traditional pulse oxymeter is that the oxymeter has to be fixed on the finger of the patient so that any movement of the patient's hand may detach the detector from the finger to invalidate the measurement. In addition, contraction in tip blood vessels of the patient's fingers caused by the variation of the ambient temperature may decrease the strength of the signal.

### BRIEF SUMMARY OF THE INVENTION

The objective of the present invention is to provide a non-invasive apparatus for continuous measuring a plurality of physiological variables from a user's ear.

In order to achieve the above-mentioned objective, and avoid the problems of the prior art, the present invention provides a non-invasive apparatus for measuring a plurality of physiological variable from a user's ear. The non-invasive apparatus comprises a sensing device, a plastic housing encapsulating the sensing device, a fastener connected to the plastic housing, and a light receiver. The plastic housing is made of a material selected from the group consisting of resin, wax, silicon-containing compound and the mixture thereof, and can be deformed in accordance with the contour of an auditory meatus of the user. Particularly, the plastic housing includes an awl-shaped portion capable of being inserted into the auditory meatus and a protrusion capable of engaging with a triangular fossa of the user's ear.

The fastener can be a flexible tube, which is deformable in accordance with the shape of a helix of the user' ear to engage with the helix. The non-invasive apparatus can be positioned on the user's ear by engaging the fastener with the helix and engaging the protrusion with the triangular fossa. In addition, an opaque tape can be optionally used to further adhere the non-invasive apparatus onto the ear to avoid the non-invasive apparatus departing from the ear, and to prevent the non-invasive apparatus from being disturbed by the environment.

The sensing device comprises a body with an inner end, a temperature detector positioned at the inner end and a light emitting device positioned on the body. The temperature detector can be a thermistor aiming exactly at the tympanic membrane for measuring the user's body temperature from the tympanic membrane. The temperature detector is preferably positioned on the awl-shaped portion of the plastic housing with a wax coating on the surface. The wax coating will be softened by the user's body heat as the temperature detector approaches the tympanic membrane so that the temperature detector is allowed to precisely measure the user's body temperature without causing discomfort to the user.

The light emitting device comprises at least one light source positioned preferably at the inner side of the tragus, while the light receiver comprises a light detector positioned at the outer side of the tragus or vice versa. The light source of the light emitting device can emit a light beam to the tragus, and the light receiver can receive the light beam penetrating through the tragus. Consequently, the light emitting device incorporating the light receiver can detect the blood oxygen saturation by measuring the energy loss of the light beam due to the penetration through the tragus, i.e., the absorbency of the light beam by blood vessels in the tragus.

Compared with prior art measuring the user's body temperature from the ear and the blood oxygen saturation from the finger, respectively, the present invention non-invasive apparatus can continuously measure a plurality of physiological variables from the user's ear along. Since the plastic housing can be automatically deformed by the user's body temperature to match with the contour of the auditory meatus, it will not cause discomfort to the user and can be fixed on the user's ear for a long period of time to measure the blood oxygen saturation from the tragus and body temperature from the tympanic membrane.

In addition, measuring the blood oxygen saturation from the tragus has the following advantage:
1. Since the blood within the tragus comes from the superficial temporal artery, which extends from the main artery through the carotid artery all the way to the ear, the blood within the tragus directly comes from the heart, and possesses the correct blood oxygen saturation information.
2. The blood vessels within the tragus does not shrink as the temperature of the environment varies, therefore they possess a stable physiological signal than the other vessels.

### BRIEF DESCRIPTION OF THE SEVERAL VIEWS OF THE DRAWINGS

Other objectives and advantages of the present invention will become apparent upon reading the following description and upon reference to the accompanying drawings in which:

FIG. 1(a) and FIG. 1(b) illustrate the conformation of a user's ear;

FIG. 2 illustrates a non-invasive apparatus for measuring physiological variables according to the first embodiment of the present invention;

FIG. 3 illustrates a sensing device according to the first embodiment of present invention;

FIG. 4 illustrates a non-invasive apparatus for measuring physiological variables according to the second embodiment of present invention; and

FIG. 5 illustrates a sensing apparatus according to the second embodiment of the present invention.

### DETAILED DESCRIPTION OF THE INVENTION

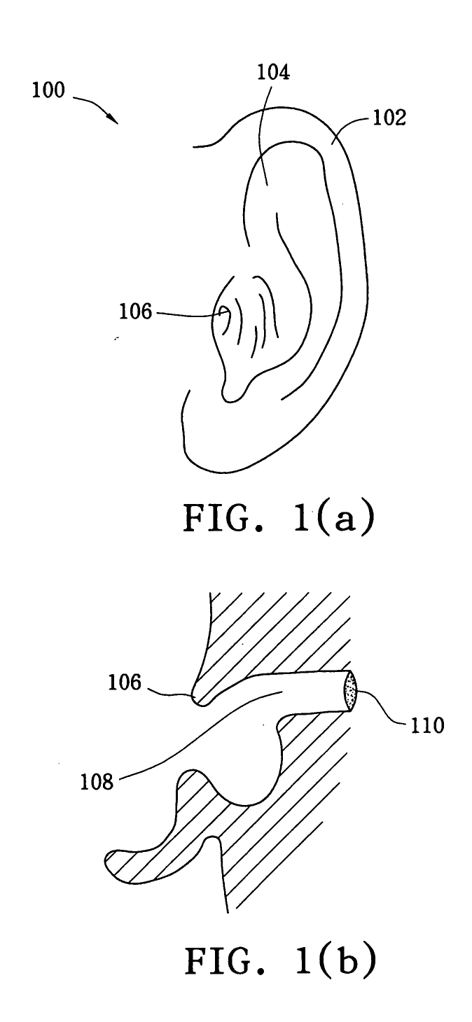

FIG. 1(a) and FIG. 1(b) illustrate the conformation of a user's ear 100. The ear 100 includes a helix 102, a triangular fossa 104, a tragus 106, an auditory meatus 108 and a tympanic membrane 110.

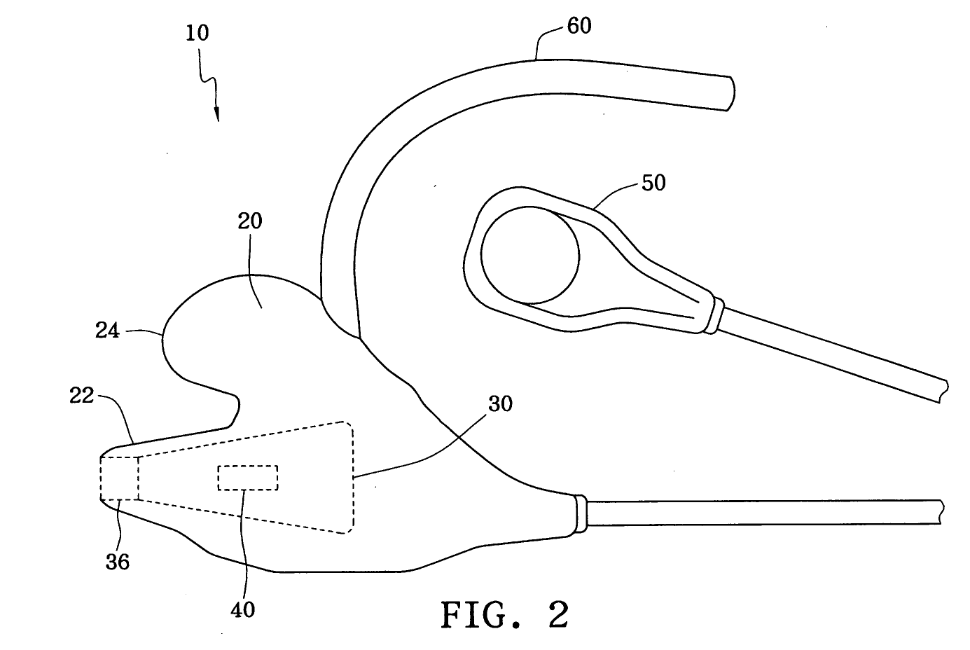

FIG. 2 illustrates a non-invasive apparatus 10 for measuring physiological variables according to the first embodiment of the present invention. As shown in FIG. 2, the non-invasive apparatus 10 comprises a sensing device 30, a plastic housing 20 encapsulating the sensing device 30, a fastener 60 connected to the plastic housing 20, and a light receiver 50. The fastener 60 can be a flexible tube, which is deformable in accordance with the shape of the helix 102 to engage with the helix 102. Particularly, the plastic housing 20 includes an awl-shaped portion 22 capable of inserting into the auditory meatus 108 and a protrusion 24 capable of engaging with the triangular fossa 104. Moreover, an opaque tape can be optionally used to further adhere the non-invasive apparatus 10 onto the ear 100 to avoid the non-invasive apparatus 10 departing from the ear 100, and to prevent the non-invasive apparatus 10 from being disturbed by the environment.

The plastic housing 20 can be deformed in accordance with the contour of the auditory meatus 108, and softened when the ambient temperature is above 33° C. Namely, the plastic housing 20 can be softened and deformed into any shape by the user's body temperature (about 37° C.), without a molding process. Moreover, although there may be some difference between the shape of the plastic housing 20 and the contour of the helix 108 before the plastic housing 20 is inserted into the user's ear 100, the body temperature at 37° C. will automatically heat and soften the unfit position of the plastic housing 20 to make it match with the contour of the helix 108 exactly so that the insertion of the non-invasive apparatus 10 will not cause discomforts.

The plastic housing 20 is made of organic materials selected from the group consisting of resin, wax, silicon-containing compound and the mixture thereof. The resin used in the plastic housing 20 substantially contains carbon, nitrogen and oxygen, and the content of resin in the plastic housing 20 is in a range between 40 and 60 wt %. The content of wax in the plastic housing 20 is in a range between 15 and 35 wt %, and wax will soften when the temperature is above 28° C. The content of silicon-containing material in the plastic housing 20 is in the range between 25 and 50 wt %, and primarily functions to modulate the hardness of the plastic housing 20.

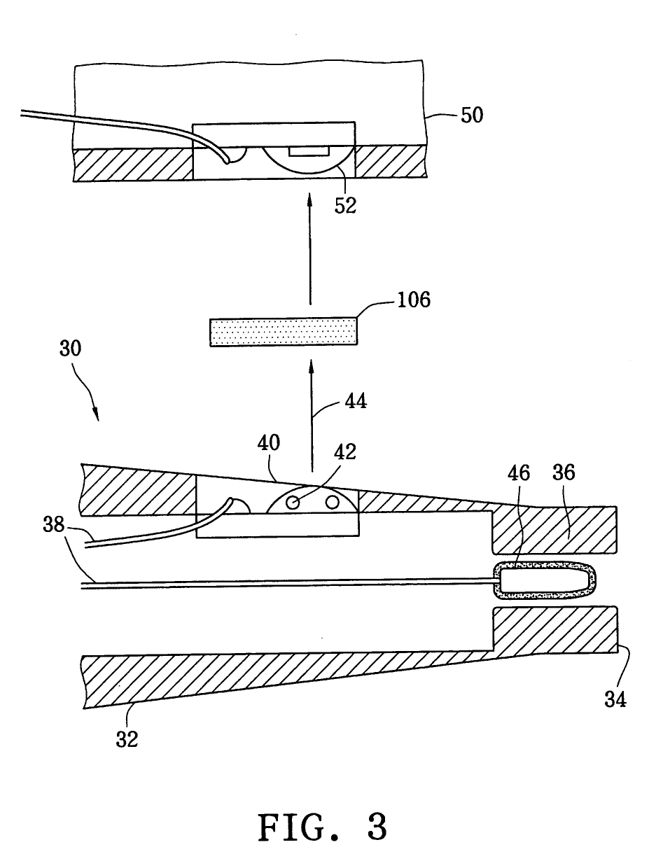

FIG. 3 illustrates the sensing device 30 according to the first embodiment of present invention. The sensing device 30 comprises a body 32 with an inner end 34, a temperature detector 36 coated with an organic material 46 positioned in the body 32 in a movable manner, a light emitting device 40 positioned on the body 32 and several wires 38 for transmitting signals. The temperature detector 36 can be a thermistor, which can move in the body 32 to contact the tympanic membrane 110 for measuring the user's body temperature from the tympanic membrane 110. Preferably, the thermistor is coated with organic coating, which will be softened and deformed to prevent the auditory meatus and the tympanic membrane from trauma.

The light emitting device 40 comprises at least one light source 42 positioned preferably at the inner side of the tragus 106, while the light receiver 50 comprises a light detector 52 positioned at the outer side of the tragus 106. The light source 42 of the light emitting device 40 can emit a light beam 44 to the tragus 106, and the light receiver 50 can receive the light beam 44 penetrating through the tragus 106. Consequently, the light emitting device 40 incorporating the light receiver 50 can measure the blood oxygen saturation by detecting the energy loss of the light beam 44 due to the penetration through the tragus 106, i.e., absorbency of the light beam 44 by blood vessels in tragus 106.

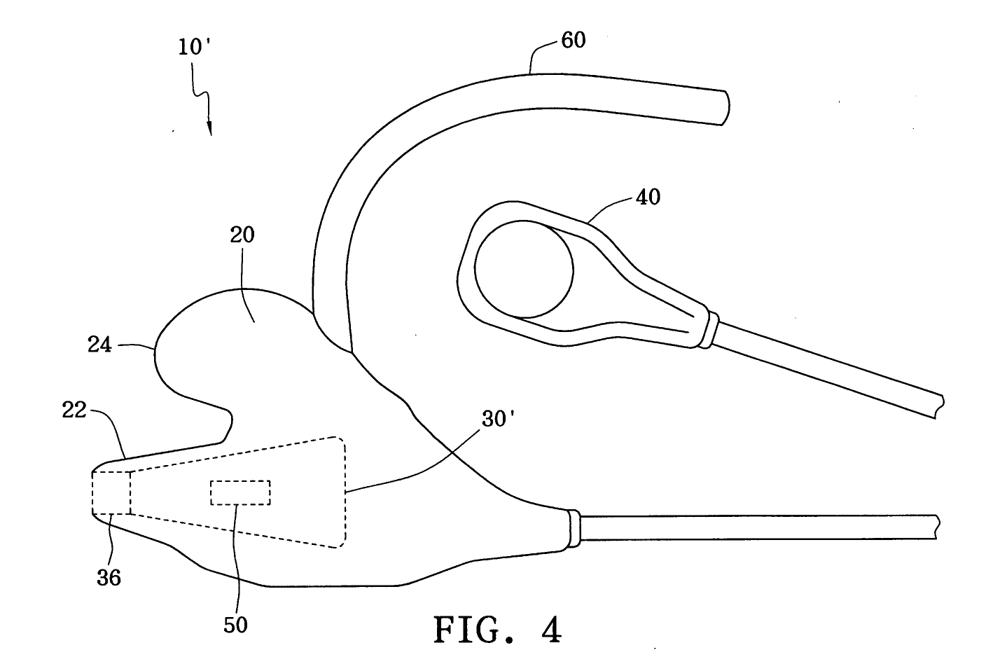

FIG. 4 illustrates a non-invasive apparatus 10′ for measuring physiological variables according to the second embodiment of present invention. Compared with the non-invasive apparatus 10 illustrated in FIG. 2, the non-invasive apparatus 10′ exchanges the position of the light receiver 50 with that of the light emitting device 40. Namely, the light receiver 50 of the non-invasive apparatus 10′ is positioned on the sensing device 30′ inside the plastic housing 20, while the light emitting device 40 is positioned outside the plastic housing 20.

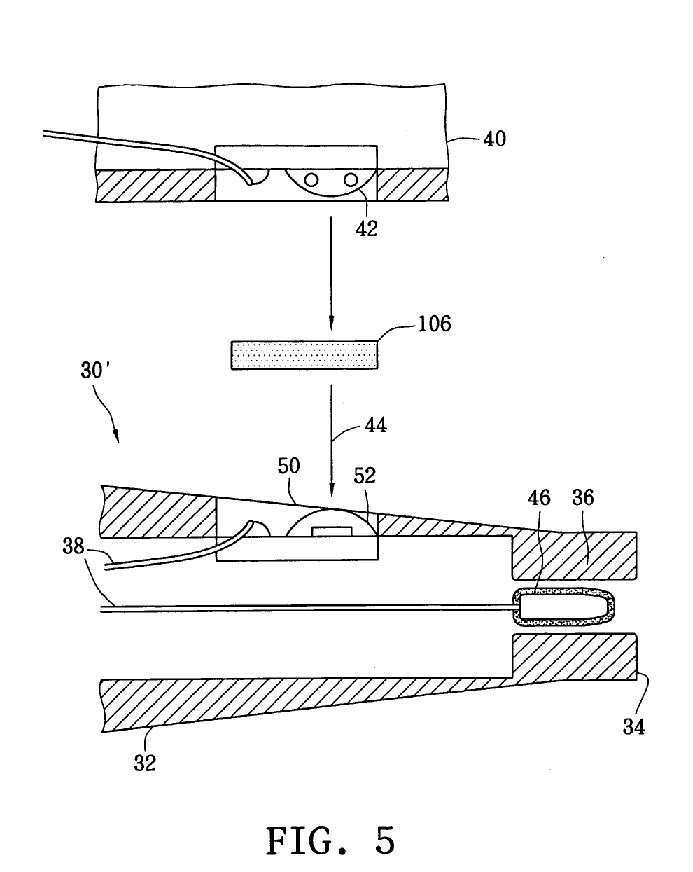

FIG. 5 illustrates the sensing apparatus 30′ according to the second embodiment of the present invention. As shown in FIG. 5, the light emitting 40 emits a light beam 44 to the tragus 106 from the outer side of the tragus 106, and the light receiver 50 positioned at the inner side of the tragus 106 detects the luminous intensity of the light beam 44 penetrating through the tragus 106. Consequently, the light emitting device 40 incorporating the light receiver 50 can measure the blood oxygen saturation by detecting the energy loss of the light beam 44 due to the penetration through the tragus 106, i.e., absorbency of the light beam 44 by blood vessels in tragus 106.

Compared with prior art, the present non-invasive apparatus 10 for measuring physiological variables possesses the following advantages:
1. The plastic housing 20 can be automatically deformed by the user's body heat to match with the contour of auditory meatus 108, so that it will not cause discomfort to the user and can be fixed on the user's ear 100 for a long period of time to provide consecutive body temperature data.
2. The prior art measures body temperature from the ear and measures the blood oxygen saturation from the finger, respectively. The present invention incorporates thermometer and oxymeter into a unitary non-invasive apparatus 10 for measuring physiological variable, which measures blood oxygen saturation from the tragus 106 and body temperature from the tympanic membrane 110.

The above-described embodiments of the present invention are intended to be illustrative only. Numerous alternative embodiments may be devised by those skilled in the art without departing from the scope of the following claims.

1. A non-invasive apparatus for measuring physiological variables, comprising:
a body;
a plastic housing encapsulating the body and being deformable in accordance with a contour of an auditory meatus of a user;
a temperature detector coated with an organic material positioned in the body in a movable manner, contacting a tympanic membrane of the user;
a light emitting device positioned on the body, emitting a light beam to a tragus of the user; and
a light receiver, receiving the light beam penetrating through the tragus.

2. The non-invasive apparatus for measuring physiological variables of claim 1, wherein the light emitting device is positioned at the inner side of the tragus, and the light receiver is positioned at the outer side of the tragus.

3. The non-invasive apparatus for measuring physiological variables of claim 1, further comprising.
a fastener connected to said plastic housing, fastening onto an ear of the user.

4. The non-invasive apparatus for measuring physiological variables of claim 3, wherein the fastener is comprised of a plastic tube deformable in accordance with the shape of a helix of the user to flexibly engage within the helix.

5. The non-invasive apparatus for measuring physiological variables of claim 3, further comprising
an opaque tape adhering onto the ear to prevent departing from the ear and being disturbed by environment.

6. The non-invasive apparatus for measuring physiological variables of claim 1, wherein the plastic housing is comprised of organic materials.

7. The non-invasive apparatus for measuring physiological variables of claim 1, wherein the plastic housing is comprised of materials selected from the group consisting of resin, wax, silicon-containing compound and mixture thereof.

8. The non-invasive apparatus for measuring physiological variables of claim 1, wherein the plastic housing comprises an awl-shaped portion capable of inserting into the auditory meatus, and the temperature detector is positioned on the awl-shaped portion.

9. The non-invasive apparatus for measuring physiological variables of claim 1, wherein the plastic housing comprises a protrusion capable of engaging with a triangular fossa of the user.

10. The non-invasive apparatus for measuring physiological variables of claim 1, said temperature detector coated with an organic material, said organic material being selected from the group consisting of resin, wax, silicon-containing compound and mixture thereof.

11. A non-invasive apparatus for measuring physiological variables, comprising:
a body;
a plastic housing encapsulating the body and being deformable in accordance with contour of an auditory meatus of a user;
a temperature detector coated with an organic material positioned in the body in a movable manner, contacting a tympanic membrane of the user;
a light emitting device emitting a light beam to a tragus of the user; and
a light receiver positioned on the body detecting the light beam penetrating through the tragus.

12. The non-invasive apparatus for measuring physiological variables of claim 11, wherein the light receiver is positioned at the inner side of the tragus, and the light emitting device is positioned at the outer side of the tragus.

13. The non-invasive apparatus for measuring physiological variables of claim 11, further comprising:
a fastener connected to the plastic housing for fastening onto an ear of the user.

14. The non-invasive apparatus for measuring physiological variables of claim 13, wherein the fastener is comprised of a plastic tube deformable in accordance with the shape of a helix of the user to flexibly engage within the helix.

15. The non-invasive apparatus for measuring physiological variables of claim 13, further comprising:
an opaque tape adhering onto the ear to prevent departing from the ear and being disturbed by environment.

16. The non-invasive apparatus for measuring physiological variables of claim 11, wherein the plastic housing is comprised of organic materials.

17. The non-invasive apparatus for measuring physiological variables of claim 11, wherein the plastic housing is comprised of materials selected from the group consisting of resin, wax, silicon-containing compound and mixture thereof.

18. The non-invasive apparatus for measuring physiological variables of claim 11, wherein the plastic housing comprises an awl-shaped portion capable of inserting into the auditory meatus, and the temperature detector is positioned on the awl-shaped portion.

19. The non-invasive apparatus for measuring physiological variables of claim 11, wherein the plastic housing comprises a protrusion capable of engaging with a triangular fossa of the user.

20. The non-invasive apparatus for measuring physiological variables of claim 11, said temperature detector coated with an organic material, said organic material being selected from the group consisting of resin, wax, silicon-containing compound and mixture thereof.
